(19) United States
(12) Patent Application Publication
(10) Pub. No.: US 2004/0199107 A1
(43) Pub. Date: Oct. 7, 2004

(54) Breast milk pump

(75) Inventor: Hansueli Nuesch, Remetschwil (CH)

Correspondence Address: BURNS DOANE SWECKER & MATHIS L L P, POST OFFICE BOX 1404, ALEXANDRIA, VA, 22313-1404, US

(73) Assignee: Nuesch Engineering

(21) Appl. No.: 10/406,595

(22) Filed: Apr. 4, 2003

(51) Int. Cl. A61M 1/06

(52) U.S. Cl. 604/74

(57) ABSTRACT

The invention relates to a breast-milk pump containing a breast cup, a suction connection that can be connected to the suction pump and a blocking device which seals the suction connection against the entry of milk. The pump is provided with a valve housing, containing an inlet that leads to the breast cup, an opening connected to the suction pump and a valve body. The valve body contains a cladding surface and a closing surface, which together form a hollow body, whose opening faces towards the inlet. The hollow body is mounted on a support so that one side can freely pivot. If during a suction phase, the level of milk that has risen above the collection container reaches the inlet, a stream of milk enters the hollow body. As a result of the deflection of the stream against the closing surface, the valve part receives a pressure surge or impulse, which is sufficient to displace the closing surface against the connection opening, where the closing surface remains during the additional suction phase, thus protecting the suction connection against the entry of milk. The valve body has a compact construction and guarantees a secure seal.

### RELATED APPLICATIONS

This application claims priority under 35 U.S.C. §119 to EP Application 00121825.4 filed in the EPO on Oct. 6, 2000, and under 35 U.S.C. §120 to PCT/CH01/00555 filed as an International Application on 14 Sep. 2001, the entire contents of which are hereby incorporated by reference in their entireties.

### BACKGROUND

The invention relates to a breast milk pump. A breast milk pump is known from DE-A-197 47 842. Therein, a housing is provided having an inlet opening at one end and a valve opening at the other end. Opposite to the inlet opening is the front surface of an elongated valve body for overflow protection so that in the case of milk entering the housing, it first impinges on this surface so that the valve body closes the opposite valve opening before the milk, passing the valve body, had time enough to reach the region of the valve opening. In this design, the valve body is suspended on a carrier membrane which guides the valve body to a linear movement towards the valve opening by arms either being arranged in opposing relationship or in a star-shape around the valve body.

In practice, this arrangement proved to work quite well, because milk entering the pumping unit would damage it to such an extent that it had to be exchanged. Practice, however, has shown that the valve body, and thus the housing too, has to be formed by two parts and is relatively long. This makes production of the valve expensive and, in addition, renders it relatively space consuming.

### SUMMARY

The invention is directed to forming a valve of the type mentioned at the beginning in such a way that safe closure is also possible with a smaller construction.

In this way, a flow-dynamic effect is created, on the one hand, which could not have been simply expected: Entering milk splashes through the inlet opening against the upper front surface of the hollow valve body and sets it into its closure position. Even if the valve body, as compared with the prior art, is relatively short, the stretch of the milk is relative long, because it is downwards deviated by the upper front surface of the hollow body and has to rise again in order to reach the valve opening. This means that the way of the milk is folded from the inner side to the outer side. Furthermore, the hollow valve body, whose lower side is open, has only a small volume so that relatively much milk is received in the region of the valve body up to the moment where this region is filled with milk up to the valve.

In addition, yet another favorable effect-has been shown: by the fact that the valve body is hollow and a lower front surface, which up to now was decisive for the striking of the milk, is omitted, and, moreover, the whole valve body is shorter, it has a smaller inertia. If one considers the unit of a, preferably membrane like, carrier and valve body as a mass-spring system able to vibrate, it will be clear that the inherent frequency of the system increases with decrease of the mass, which means that the valve according to the invention can close still faster than that of the prior art and, thus, can even be built still smaller.

Furthermore, another effect is that the former linear guidance by several arms of the carrier resulted in a certain stiffness. By providing a unilateral articulation (for example, the carrier being formed by a flexible body, particularly by a membrane as known per se, but optionally can be a rigid body having a hinge), mobility can be enhanced, and the above effect of acceleration of response can be more significant.

Although the opening of the hollow body, open to the lower side, can have any dimension, the above-mentioned favorable flow dynamics can result, if the opening has the predetermined cross-sectional area so as to be aligned with the outer surface of the hollow space.

Further acceleration results and function are ensured, if at least part of the unit, which includes valve body and the carrier, is made of a material having a smaller density than 1.034, preferably less than about 1.028. The idea is that by selecting such a material a certain floating effect is utilized, because breast milk will ordinarily have a density of about 1.034, in rare cases even of 1.028. Thus, a strong buoyancy is ensured still when entering milk reaches the unit.

An exemplary material for achieving this effect is silicone rubber, because this material is also suitable for food and can be easily cleaned, if necessary.

### BRIEF DESCRIPTION OF THE DRAWINGS

Further details of the invention will become apparent of the following description of a particularly preferred embodiment schematically illustrated in the drawings in which:

FIG. 1 is an axial cross-section of a unit including a breast cup and suction connection as well as an interposed valve;

FIG. 2 is an illustration enlarged in comparison with FIG. 1 which shows the valve at the suction connection; and

FIGS. 3 and 4 represent the unit of carrier membrane and hollow valve body each in a perspective view from above and below.

### DETAILED DESCRIPTION OF THE PREFERRED EMBODIMENTS

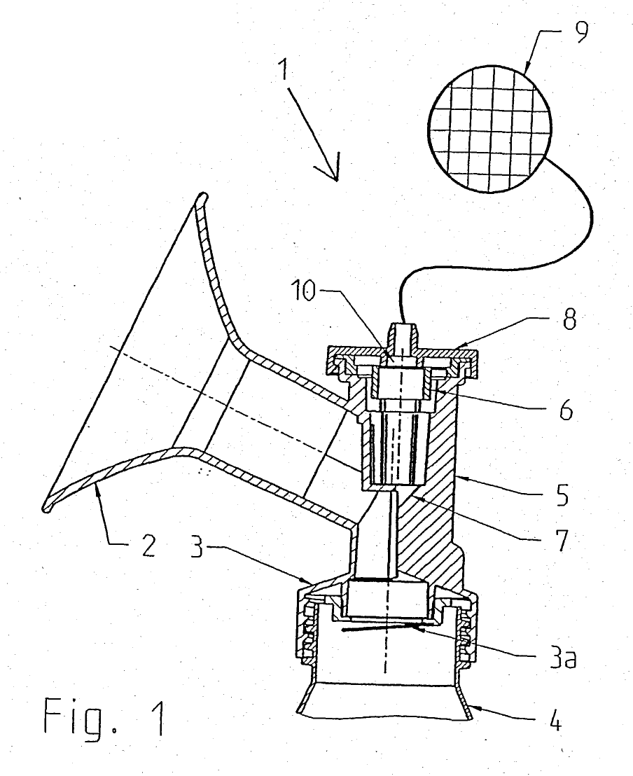

FIG. 1 shows a breast milk pump 1 comprising a breast cup 2, a connection arrangement 3 to a collection container 4 to be screwed to it, and a valve housing 5 wherein a valve body 6 is movably arranged. A non-return valve 3a can be assigned to the connection arrangement 3 that closes during the suction phases and through which breast milk can flow from the breast cup 2 to the collection container 4. The interior of the valve housing 5 is connected to the breast cup 2 through an inlet opening 7 on a first front surface of the valve housing 5, and through a suction connection 8 to a suction pump 9. In order to prevent that milk is sucked into the suction pump 9, the valve body 6 can be displaced towards a connection opening 10 of the suction connection 8 by milk entering the valve housing 5 through the inlet opening 7.

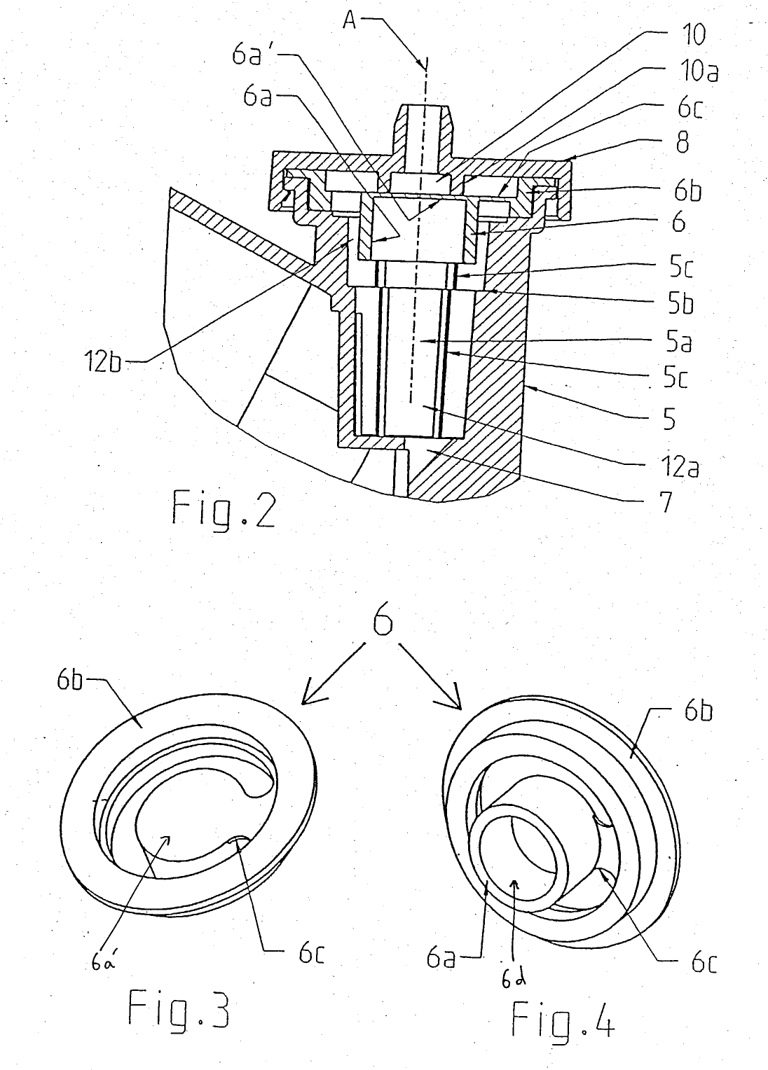

According to FIG. 2, the valve housing 5 comprises an inner space 5a that extends along an axis A from the inlet opening 7 to a valve, or connection, opening 10. In the embodiment shown, the cross-section of the inner space 5a enlarges from the inlet opening 7 towards the connection opening 10. The enlargement can include an enlarging shoulder 5b formed between the inlet opening 7 and the valve body 6. In this way, the outer dimension of the valve body 6 can be selected substantially in correspondence with the smaller cross-section at the enlarging shoulder 5b without the possibility of the valve body 6 jamming in the inner space. If milk enters the inner space 5a in a flood, milk will impinge on the valve body 6. Due to the enlarging shoulder, it is quite improbable that it can reach directly the annular space around the valve body 6.

The valve body 6 comprises a cylindrical valve portion having a cladding surface 6a and a closing surface 6a′, a carrier 6b and a connection portion 6c. The cylindrical valve portion forms a hollow body having an opening 6d of, for example, predetermined cross-section which faces the inlet opening 7. The closing surface 6a′ is assigned to the connection opening 10, and the cylindrical valve portion represented as cladding surface 6a and closing surface 6a′ are pivotally connected to the carrier 6b via a movable interconnection 6c. Starting from the inlet opening 7, a first connection channel section 12a leads to the valve body 6. In the region of the valve body 6, an annular space 12b is formed as a second connection channel section between the valve body 6 and the valve housing 5. In order to ensure passage of air without any problem even with the cylindrical valve portion in inclined position, optionally longitudinal ribs 5c may formed at the inner surface of the valve housing 5.

According to FIGS. 3 and 4, a valve body 6 is formed in one piece. The carrier 6b is ring-shaped and fits into a receiving space between the valve housing 5 and the suction connection 8. By attaching the suction connection 8 to the valve housing 5, the carrier is firmly clamped. The cladding surface 6a and the closing surface 6a′ are connected to the carrier 6b through the connection portion 6c, at least the connection portion 6c being of an elastic material so that a pivoting movement can be ensured about a pivoting axis of articulation extending, for example, between the rounded edges of the connection portions 6c. At least part of the unit, which contains the valve body 6 and the carrier 6b, is made from a material of a smaller density than 1.034 kgm⁻³, preferably less than 1.028 kgm⁻³, or of the density of the material being collected. The idea is that by selecting such a material a certain floating effect is utilized. For example, breast milk will ordinarily have a density of about 1.034, in rare cases even of 1.028.

In order to create the possibility of closing the connection opening 10 when moving the valve body 6 towards the connection opening 10, an annular portion 10a projects from the edge of the connection opening 10 in axial direction up to a plane of the non-deviated closing surface 6a′ and comprising the connection portion 6c. When pumping milk, the valve housing 5 is oriented in a direction that the cladding surface 6a together with the closing surface 6a′ is somewhat downwards deviated due to its own weight with the connection portion 6c being slightly bent. The deviation results in that a suction connection from the connection opening 10 to the inlet opening 7 is open to be passed through.

If now the level of milk is rising above the collection container and reaches the inlet opening 7 during a suction phase, a jet of milk enters the interior of that valve portion which includes the cladding surface 6a and the closing surface 6a′. By deviating the jet at the closing surface 6a′, the valve portion 6a, 6a′ obtains an impact force or pressure that is sufficient to move the closing surface 6a′ to the annular portion 10a where the closing surface 6a′ adheres during a suction phase, thus preventing the milk from being sucked through the connection opening 10. In case the pressure impact and the suction effect were not sufficient to close the connection opening 10, the milk in the connection channel 12a, 12b provokes an increased flow resistance during further suction cycles and a buoyant force acts onto the valve body, both these effects resulting in such a movement of the valve portion 6a, 6a′ as necessary for closing the connection opening 10. If milk were rising in the valve housing 5 up to the cladding surface 6a, the partially air filled hollow space surrounded by the cladding surface 6a will result in a strong buoyancy of the valve portion and, thus, presses the closing surface 6a′ against the opening's annular portion 10a. Since the lower area of the closing surface 6a′ is used as a striking surface for the entering milk flood rather than a surface at the inlet opening 7, a large volume can be provided in the valve housing 5 which receives the milk before it reaches the connection opening 10. In addition, the valve body 6 can be formed with a short peripheral shell and be extremely lightweight so that already a small milk jet provokes an impact force necessary for blocking. The enlarging shoulder 5b and an optimum choice of the cross-sectional area of the valve body can reduce a risk of the milk jet taking the direct way from the inlet opening 7, through the connection channel 12a, 12b to the connection opening 10. Optionally, the enlarging shoulder 5b is formed so large that the cross-sectional sectional area of the inner space 5a is smaller than the cross-sectional area of the cladding surface 6a. The valve body is small and, can be formed in one piece, thus ensuring safe closure.

In breast milk pumps for two breast cups, an overflow protection comprising a valve arrangement can be assigned to each one of the breast cups. Optionally, however, one overflow protection can be sufficient which is connected to a common interconnection portion of both breast cups.

The overflow protection developed for a breast milk pump and comprising the valve housing 5 and the valve body 6 moveable therein can also be used with advantage for other medical suction apparatuses.

It will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

1. Breast milk pump comprising:
at least one breast cup;
a suction connection that can be connected to a suction pump; and
a blocking device for milk which includes a valve body having a cladding surface and which is axially moveably held by a carrier at a valve opening within a valve housing which is provided with an inlet opening approximately opposite the valve body and is between a connection arrangement joining the at least one breast cup for draining milk into a collection container, wherein the valve body is formed as a hollow body having an opening that faces the inlet opening with a hollow space of predetermined cross-section, which is defined by the cladding surface, and said valve body being unilaterally freely pivotal on its carrier about an axis of articulation.

2. Breast milk pump according to claim 1, wherein the opening has the predetermined cross-section aligned with the cladding surface of the hollow space.

3. Breast milk pump according to claim 1, wherein at least said valve body and said carrier are formed of a material having a smaller density than 1.034.

4. Breast milk pump according to claim 3, wherein at least said valve body and said carrier are formed of silicone rubber.

5. Valve for a breast milk pump comprising:
a valve body having a cladding surface that is axially moveably held by means of a carrier at a valve opening in a valve housing which is provided with an inlet opening at an end which faces said valve body, the valve body being formed as a hollow body having an opening that faces the inlet opening, a hollow space of the hollow body having a predetermined cross-section which is defined by the cladding surface, and said valve body freely pivotal on its carrier about an axis of articulation.

6. Valve according to claim 5, wherein the opening has the predetermined cross-sectional aligned with the cladding surface of the hollow space.

7. Valve according to claim 5, wherein at least said valve body and said carrier are formed of a material having a smaller density than 1.034.

8. Valve according to claim 7, wherein at least said valve body and said carrier are formed of silicone rubber.

9. Breast milk pump according to claim 1, wherein at least said valve body and said carrier are formed of a material having a smaller density than 1.028.

10. Breast milk pump according to claim 5, wherein at least said valve body and said carrier are formed of a material having a smaller density than 1.028
